(19) United States
(12) Patent Application Publication
(10) Pub. No.: US 2018/0116998 A1
(43) Pub. Date: May 3, 2018

(54) USE OF CANNABIS TO TREAT FIBROMYALGIA, METHODS AND COMPOSITIONS THEREOF

(71) Applicant: ONE WORLD CANNABIS LTD, Petach Tikva (IL)

(72) Inventors: Alon Sinai, Petach Tikva (IL); Ziv Turner, Petach Tikva (IL); Yehuda Baruch, Gedera (IL)

(73) Assignee: ONE WORLD CANNABIS LTD, Petach Tikva (IL)

(21) Appl. No.: 15/573,687

(22) PCT Filed: May 11, 2016
(86) PCT No.: PCT/IL2016/050498

§ 371 Date: Nov. 13, 2017

Related U.S. Application Data
(60) Provisional application No. 62/160,609, filed on May 13, 2015.

(51) Int. Cl. A61K 31/352; A61K 31/05; A61P 25/04; A61K 36/185

(52) U.S. Cl. CPC A61K 31/352; A61K 36/185; A61P 25/04; A61K 31/05

(57) ABSTRACT

The present invention discloses a composition comprising cannabis natural extract, or synthetic cannabinoids, for use in treating a subject suffering from fibromyalgia. The present invention further discloses methods and uses of the aforementioned composition.

### FIELD OF THE INVENTION

The current invention relates to a novel treatment for fibromyalgia. More specifically the invention pertains to a composition comprising cannabis extract or fractions thereof, or cannabis synthetic components, for treating fibromyalgia.

### BACKGROUND OF THE INVENTION

Fibromyalgia is a disorder characterized by widespread musculoskeletal pain accompanied by fatigue, sleep, memory and mood issues. Fibromyalgia syndrome is believed to be caused by amplified painful sensations caused by the way the brain processes pain signals. Symptoms sometimes begin after a physical trauma, surgery, infection or significant psychological stress. In other cases, symptoms gradually accumulate over time with no single triggering event.

Women are much more likely to develop fibromyalgia than are men. Many people who have fibromyalgia also have tension headaches, temporomandibular joint (TMJ) disorders, irritable bowel syndrome, anxiety and depression.

While there is no cure for fibromyalgia, a variety of medications can help control symptoms, as well as alterations of lifestyle habits such as exercise, relaxation and stress-reduction measures. The exact onset trigger is unknown but is believed to involve psychological, genetic, neurobiological and environmental factors, which among them stress levels are highly prominent.

The central symptom of fibromyalgia, namely widespread pain, appears to result from neuro-chemical imbalances including activation of inflammatory pathways in the brain which results in abnormalities in pain processing (Clauw D J et al., 2011, “The science of fibromyalgia”. Mayo Clin Proc 86 (9): 907-11). The brains of fibromyalgia patients show functional and structural differences from those of healthy individuals, but it is unclear whether the brain anomalies cause fibromyalgia symptoms or are the product of an unknown underlying common cause. Some research suggests that these brain anomalies may be the result of childhood stress, or prolonged or severe stress (Schweinhardt P et al., October 2008, “Fibromyalgia: a disorder of the brain?”. Neuroscientist 14 (5): 415-21).

Fibromyalgia Syndrome (FMS) is a persistent and debilitating disorder estimated to impair the quality of life of 2-4% of the population, with 9:1 female-to-male incidence ratio. FMS is the second most common disorder, after osteoarthritis, observed by rheumatologists. The defining symptoms of FMS include chronic widespread pain, intense pain in response to tactile pressure (allodynia), prolonged muscle spasms, weakness in the limbs, nerve pain, muscle twitching, palpitations and diffuse tenderness, along with fatigue, sleep disturbance and cognitive impairments. These impairments include problems with short- and long-term memory, short-term memory consolidation, impaired speed of information processing, reduced attention span and limited multi-tasking performance. FMS is a persistent disorder with symptoms that have a devastating effect on people's lives, including limited ability to engage in everyday activities, limited ability to maintain outside work and difficulties to maintain normal relationships with family, friends and employers. These limitations can lead to the occurrence of anxiety and depression in many FMS patients.

FMS is not completely understood, in part because there is no evidence of a single event that “causes” fibromyalgia. Rather, many physical and/or emotional stressors may trigger or aggravate symptoms. Those have included certain infections, such as a viral illness or Lyme disease, as well as emotional or physical trauma. Establishing proper diagnostic criteria is also a challenge.

As with many other syndromes, there is no efficient cure for FMS and no agreed upon treatment—the suggested treatment depends on the classification of choice. Those who regard FMS as a neurological disorder advocate pharmacotherapy. All current treatments, such as prescribed medications, aerobic exercises and cognitive behavioral therapies, consist of symptom management. Integrated programs based on these treatments have been shown to alleviate pain and some other symptoms but with limited effectiveness.

Fibromyalgia is currently treated with pain and depression management drugs. However, due to the origins of the disorder in the brain, and its association with high stress levels, there is a call for a medication that will enable both stress reduction and brain function modification.

Previous trials have shown that both naturally occurring and endogenous cannabinoids hold analgesic qualities, particularly in the treatment of pain resistant to conventional pain therapies.

In view of the above there is a long felt and unmet need for a naturally originated pharmaceutical composition that is specifically useful for treatment of fibromyalgia and chronic pain symptoms.

### SUMMARY OF THE INVENTION

It is thus one object of the present invention to provide a composition comprising a therapeutically effective amount of Tetrahydrocannabinol (THC) or a derivative thereof, or Cannabidiol (CBD) or a derivative thereof, or a combination thereof for use in relieving a subject suffering from fibromyalgia syndrome symptoms.

It is also an object of the present invention to provide the aforementioned composition, wherein said Tetrahydrocannabinol (THC) or a derivative thereof, or said Cannabidiol (CBD) or a derivative thereof is from cannabis extract.

It is also an object of the present invention to provide the aforementioned composition comprising a therapeutically effective amount of cannabis extract for use in relieving a subject suffering from fibromyalgia syndrome symptoms.

It is also an object of the present invention to provide the aforementioned composition, wherein the cannabis extract contains tetrahydrocannabinol (THC) or a derivative thereof, or cannabidiol (CBD) or a derivative thereof, or a combination thereof.

It is also an object of the present invention to provide the aforementioned composition, wherein the THC or a derivative thereof is selected from the group consisting of THC, Tetrahydrocannabivarin (THCV), Tetrahydrocannabinolic acid (THCA) and any combination thereof.

It is also an object of the present invention to provide the aforementioned composition, further comprising THC or a derivative thereof selected from the group consisting of natural THC or a derivative thereof produced in the body of humans and animals, THC or a derivative thereof extracted from plants, synthetic THC or a derivative thereof, and any combination thereof.

It is also an object of the present invention to provide the aforementioned composition, wherein the THC or a derivative thereof is extracted from cannabis; the cannabis is selected from the group comprising of: Cannabis sativa, Cannabis indica, Cannabis ruderalis, and any combination thereof.

It is also an object of the present invention to provide the aforementioned composition, wherein the cannabidiol (CBD) or a derivative thereof is selected from the group consisting of CBD, cannabidivarin (CBDV), cannabidiolic acid (CBDA) and any combination thereof.

It is also an object of the present invention to provide the aforementioned composition, further comprising CBD or a derivative thereof selected from the group consisting of natural CBD or a derivative thereof produced in the body of humans and animals, CBD or a derivative thereof extracted from plants, synthetic CBD or a derivative thereof, and any combination thereof.

It is also an object of the present invention to provide the aforementioned composition, wherein the CBD or a derivative thereof is extracted from cannabis; the cannabis is selected from a group consisting of: Cannabis sativa, Cannabis indica, Cannabis ruderalis, and any combination thereof.

It is also an object of the present invention to provide the aforementioned composition, wherein the concentration of the THC is in the range of about 2% to about 85%.

It is also an object of the present invention to provide the aforementioned composition, wherein the concentration of the CBD is in the range of about 2% to about 85%.

It is also an object of the present invention to provide the aforementioned composition, further comprising a ratio of the THC, or a derivative thereof, to the CBD, or a derivative thereof, of 4:1 or 5:1, respectively.

It is also an object of the present invention to provide the aforementioned composition, wherein the composition is formulated for administration of between about 10 mg THC and about 160 mg THC per day, preferably between about 10 mg and about 100 mg THC per day.

It is also an object of the present invention to provide the aforementioned composition, wherein the composition is formulated for administration of between about 5 mg THC and about 20 mg THC per dosage unit, preferably about 10 mg THC per dosage unit.

It is also an object of the present invention to provide the aforementioned composition, wherein the composition is formulated for administration of between about 2 mg CBD and about 40 mg CBD per day, preferably between about 2 mg and about 20 mg CBD per day.

It is also an object of the present invention to provide the aforementioned composition, wherein the composition is formulated for administration of between about 1 mg CBD and about 5 mg CBD per dosage unit, preferably about 2 mg CBD per dosage unit.

It is also an object of the present invention to provide the aforementioned composition, wherein the composition is formulated for administration of between about 1 to about 10 times per day.

It is also an object of the present invention to provide the aforementioned composition, wherein the composition is administered in a manner selected from the group consisting of: intranasal, topical, transdermal, intravenous, oral, and any combination thereof.

It is also an object of the present invention to provide the aforementioned composition, wherein the composition is formulated in the form of drops.

It is also an object of the present invention to provide the aforementioned composition, wherein the composition is formulated in a dosage form selected from the group consisting of oil, liquid, solid, gas, oral, pill, tablet, capsule, buccal, sub-lingual, orally-disintegrating, thin film, liquid solution, suspension, powder or liquid or solid crystals, pastes, inhalational, aerosol, inhaler, nebulizer, smoking, vaporizer, parenteral, intradermal, intramuscular, intraosseous, intraperitoneal, intravenous, subcutaneous, topical, cream, gel, liniment or balm, lotion, ointment, drops, skin patch, vaginal, suppository, pessary, rectal and any combination thereof.

It is also an object of the present invention to provide the aforementioned composition, wherein the composition is administered in combination with at least one therapeutic agent selected from the group comprising of dypirone, pregabalin, duloxetine, milnacipran, tricyclic antidepressants, amitriptyline, gabapentin, paracetamol, tramadol, codeine, growth hormone, sodium oxybate, cyclobenzaprine, tizanidine, pramipexole, ropinirole, quercetin and any combination thereof.

It is also an object of the present invention to provide the aforementioned composition, wherein the composition provides a synergistic effect with respect to treatment of fibromyalgia when administered in combination with the therapeutic agent, as compared to the effect provided when the composition and the therapeutic agent administered seperately.

It is also an object of the present invention to provide the aforementioned composition, wherein the composition additionally comprises at least one carrier or excipient selected from the group consisting of diluents, antiadherents, binders, coatings, disintegrants, surfactants, dissolving agents, solubilising agents, bioadhesive agents, polysaccharides, polymers, copolymers, fast dissolving tablet (FDT) type excipient, bioavailability enhancing agent, Thin Film type excipient, PharmFilm type excipient, mucoadhesive type excipient, acidifying agents, probiotic agents, protective agents, antioxidants, effervescent excipient, dispersing agents flavours, colours, lubricants, glidants, sorbents, preservatives, sweeteners, and any combination thereof.

It is also an object of the present invention to provide the aforementioned composition, further comprising flavoring agents, selected from the group consisting of sugar, sucrose, sorbitol, sucralose, saccharin sodium, sodium cyclamate, aspartame, neotame, acesulfame potassium, stevioside, sodium chloride, D-limonene, citric acid and any combination thereof.

It is also an object of the present invention to provide the aforementioned composition, further comprising preservatives, selected from the group consisting of methylparabens, ethylparabens, propylparabens, butylparabens, sorbic acid, acetic acid, propionic acid, sulfites, nitrites, sodium sorbate, potassium sorbate, calcium sorbate, benzoic acid, sodium benzonate, potassium benzonate, calcium benzonate, sodium metabisulfite, propylene glycol, benzaldehyde, butylated hydroxytoluene, butylated hydroxyanisole, formaldehyde donors, essential oils, monoglyceride, phenol, mercury components and any combination thereof.

It is also an object of the present invention to provide the aforementioned composition, wherein the composition is in a sustained release dosage form or in an immediate release dosage form.

It is also an object of the present invention to provide the aforementioned composition, wherein the composition is in a sustained release dosage form selected from the group comprising of liposomes, drug polymer conjugates, microencapsulation, controlled-release tablet coating, and any combination thereof.

It is also an object of the present invention to provide the aforementioned composition, wherein the composition is used for preventing the onset of a flareup of fibromyalgia symptoms in the subject.

It is also an object of the present invention to provide the aforementioned composition, wherein the composition provides an improvement in fibromyalgia symptoms of the subject as measured by at least one pain severity scale, compared to an established baseline, control or placebo.

It is also an object of the present invention to provide the aforementioned composition, wherein the improvement in fibromyalgia symptoms is measured by an improvement in the subject's score of at least one point on the at least one pain severity scale, as compared to an established baseline or to a placebo.

It is also an object of the present invention to provide the aforementioned composition, wherein the at least one fibromyalgia severity scale is selected from the group comprising of Brief pain inventory, Sleep history questionnaire (PSQI), SF-36 Quality of life assessment, Fibromyalgia impact questionnaire (FIQ), Mini International Neuropsychiatric Interview, fibromyalgia syndrome Tenderness Assessment, Hamilton Depression Rating Scale (HDRS) and any combination thereof.

It is also an object of the present invention to provide the aforementioned composition, wherein the fibromyalgia symptoms are selected from the group comprising of widespread pain, chronic pain, cognitive difficulties, chronic muscle pain, muscle spasms, muscle tightness, fatigue, insomnia, stiffness, fibro fog, abdominal pain, bloating, nausea, constipation, diarrhea, headache, jaw and facial tenderness, anxiety, depression, numbness or tingling in the limbs, irritable bladder, feeling of swelling, painful menstruation, restless leg, dizziness, female or subject with established FMS confirmed by the 1990 ACR, and any combination thereof.

It is also an object of the present invention to provide the aforementioned composition, wherein the fibromyalgia symptoms is a sensitivity to at least one of the following: odors, noise, bright lights, medications, certain foods, and cold.

It is also an object of the present invention to provide the aforementioned composition, wherein said composition provides an improvement in fibromyalgia symptoms of said subject measured by a score on the average pain severity item of the BPI scale of above 5.

It is also an object of the present invention to provide the aforementioned composition, wherein said composition provides a reduction in BPI score from baseline to endpoint.

It is also an object of the present invention to provide the aforementioned composition, wherein said composition provides a sustained response defined as a 30% reduction from baseline to endpoint in the BPI score with a 30% reduction from baseline at least 2 weeks prior to the last, and with at least a 20% reduction from baseline at every week in between.

It is also an object of the present invention to provide the aforementioned composition, wherein said composition provides an improvement in pain severity as measured by the self-reported Brief Pain Inventory (short form) average pain severity score fibromyalgia impact questionnaire items.

It is also an object of the present invention to provide the aforementioned composition, wherein said pain severity score fibromyalgia impact questionnaire items comprising items which measure physical function, pain assessment, fatigue and distress.

It is also an object of the present invention to provide the aforementioned composition, wherein said composition provides improvement in measures selected from the group consisting of: quality of life, quality of sleep, disability, depression, anxiety and the patient global impression of change and the fibromyalgia severity score.

It is another object of the present invention to disclose a method for treating a subject suffering from fibromyalgia syndrome comprising steps of administering to the subject a composition comprising cannabis extract in a therapeutically effective dosage.

It is still an object of the present invention to disclose a method for treating a subject suffering from fibromyalgia syndrome comprising steps of administering to said subject a composition comprising comprising a therapeutically effective amount of Tetrahydrocannabinol (THC) or a derivative thereof, or Cannabidiol (CBD) or a derivative thereof, or a combination thereof in a therapeutically effective dosage.

It is still an object of the present invention to disclose the above mentioned method, additionally comprising a step of providing said Tetrahydrocannabinol (THC) or a derivative thereof, or Cannabidiol (CBD) or a derivative thereof, or a combination thereof derived from cannabis extract.

It is still an object of the present invention to disclose the above mentioned method, additionally comprising a step of formulating the cannabis extract to contain tetrahydrocannabinol (THC) or a derivative thereof, or cannabidiol (CBD) or a derivative thereof, or a combination thereof.

It is still an object of the present invention to disclose the above mentioned method, additionally comprising a step of selecting the THC or a derivative thereof from the group consisting of THC, THCV, THCA and any combination thereof.

It is still an object of the present invention to disclose the above mentioned method, additionally comprising steps of extracting the THC or a derivative thereof from Cannabis; the cannabis is selected from the group comprising of: Cannabis sativa, Cannabis indica, Cannabis ruderalis, and any combination thereof.

It is still an object of the present invention to disclose the above mentioned method, additionally comprising steps of selecting the cannabidiol (CBD) or a derivative thereof from the group consisting of CBD, CBDV, CBDA and any combination thereof.

It is still an object of the present invention to disclose the above mentioned method, additionally comprising steps of selecting the CBD from the group consisting of natural CBD or a derivative thereof produced in the body of humans and animals, CBD or a derivative thereof extracted from plants, synthetic CBD or a derivative thereof, and any combination thereof.

It is still an object of the present invention to disclose the above mentioned method, additionally comprising steps of extracting the CBD or a derivative thereof from Cannabis; the cannabis is selected from a group consisting of: Cannabis sativa, Cannabis indica, Cannabis ruderalis, and any combination thereof.

It is still an object of the present invention to disclose the above mentioned method, additionally comprising steps of providing a synergistic effect with respect to treating fibromyalgia as compared to the effect provided by the THC or a derivative thereof and by the CBD or a derivative thereof when administered separately.

It is still an object of the present invention to disclose the above mentioned method, additionally comprising steps of formulating the composition to comprise a ratio of the THC, or a derivative thereof, to the CBD, or a derivative thereof, of 4:1 or 5:1, respectively.

It is still an object of the present invention to disclose the above mentioned method, additionally comprising steps of administering the THC or a derivative thereof in a dosage of about 10 mg THC and about 160 mg THC per day, preferably between about 10 mg THC and about 100 mg THC per day.

It is still an object of the present invention to disclose the above mentioned method, additionally comprising steps of administering the THC or a derivative thereof in a dosage unit of between about 5 mg THC and about 20 mg THC, preferably about 10 mg THC per dosage unit.

It is still an object of the present invention to disclose the above mentioned method, additionally comprising steps of administering the CBD or a derivative thereof in a dosage of about 2 mg CBD and about 40 mg CBD per day, preferably between about 2 mg and about 20 mg CBD per day.

It is still an object of the present invention to disclose the above mentioned method, additionally comprising steps of administering the CBD or a derivative thereof in a dosage unit of between about 1 mg CBD and about 5 mg CBD, preferably about 2 mg CBD per dosage unit.

It is still an object of the present invention to disclose the above mentioned method, additionally comprising steps of administering the composition between about 1 to about 10 times through the day.

It is still an object of the present invention to disclose the above mentioned method, additionally comprising steps of administering the composition once, twice, three, four or five times through the day.

It is still an object of the present invention to disclose the above mentioned method, additionally comprising steps of administering the composition to the subject over a time period of about 1 day to about 6 months.

It is still an object of the present invention to disclose the above mentioned method, additionally comprising steps of performing blood tests for CBD and THC 1 hour and 2 hours after said dosage unit intake.

It is still an object of the present invention to disclose the above mentioned method, additionally comprising steps of formulating the composition in a dosage form of drops.

It is still an object of the present invention to disclose the above mentioned method, additionally comprising steps of formulating the composition in a dosage form selected from the group consisting of oil, liquid, solid, gas, oral, pill, tablet, capsule, buccal, sub-lingual, orally-disintegrating, thin film, liquid solution, suspension, powder or liquid or solid crystals, pastes, inhalational, aerosol, inhaler, nebulizer, smoking, vaporizer, parenteral, intradermal, intramuscular, intraosseous, intraperitoneal, intravenous, subcutaneous, topical, cream, gel, liniment or balm, lotion, ointment, drops, skin patch, vaginal, suppository, pessary, rectal and any combination thereof.

It is still an object of the present invention to disclose the above mentioned method, additionally comprising steps of administering the composition in combination with at least one therapeutic agent selected from the group comprising of dypirone, pregabalin, duloxetine, milnacipran, tricyclic antidepressants, amitriptyline, gabapentin, paracetamol, tramadol, codeine, growth hormone, sodium oxybate, cyclobenzaprine, tizanidine, pramipexole, ropinirole, quercetin and any combination thereof.

It is still an object of the present invention to disclose the above mentioned method, additionally comprising steps of providing a synergistic effect with respect to treatment of fibromyalgia when administering the composition in combination with the fibromyalgia therapeutic agent, as compared to the effect provided when the composition and the therapeutic agent are administered seperatly.

It is still an object of the present invention to disclose the above mentioned method, additionally comprising steps of administering the composition in a manner selected from the group consisting of: intranasal, transdermal, topical, intravenous, oral, and any combination thereof.

It is still an object of the present invention to disclose the above mentioned method, additionally comprising steps of formulating the composition with at least one carrier or excipient selected from the group consisting of diluents, antiadherents, binders, coatings, disintegrants, surfactants, dissolving agents, solubilising agents, bioadhesive agents, polysaccharides, polymers, copolymers, fast dissolving tablet (FDT) type excipient, bioavailability enhancing agent, Thin Film type excipient, PharmFilm type excipient, mucoadhesive type excipient, acidifying agents, probiotic agents, protective agents, antioxidants, effervescent excipient, dispersing agents flavours, colours, lubricants, glidants, sorbents, preservatives, sweeteners, and any combination thereof.

It is still an object of the present invention to disclose the above mentioned method, additionally comprising steps of formulating the composition with at least one flavoring agents, selected from the group consisting of sugar, sucrose, sorbitol, sucralose, saccharin sodium, sodium cyclamate, aspartame, neotame, acesulfame potassium, stevioside, sodium chloride, D-limonene, citric acid and any combination thereof.

It is still an object of the present invention to disclose the above mentioned method, additionally comprising steps of formulating the composition with at least one preservatives, selected from the group consisting of methylparabens, ethylparabens, propylparabens, butylparabens, sorbic acid, acetic acid, propionic acid, sulfites, nitrites, sodium sorbate, potassium sorbate, calcium sorbate, benzoic acid, sodium benzonate, potassium benzonate, calcium benzonate, sodium metabisulfite, propylene glycol, benzaldehyde, butylated hydroxytoluene, butylated hydroxyanisole, formaldehyde donors, essential oils, monoglyceride, phenol, mercury components and any combination thereof.

It is still an object of the present invention to disclose the above mentioned method, additionally comprising steps of formulating the composition in a sustained release dosage form or in an immediate release dosage form.

It is still an object of the present invention to disclose the above mentioned method, additionally comprising steps of formulating the composition in a sustained release dosage form; wherein the sustained release dosage form is selected from a group consisting of liposomes, drug polymer conjugates, microencapsulation, controlled-release tablet coating, and any combination thereof.

It is still an object of the present invention to disclose the above mentioned method, additionally comprising steps of preventing the onset of a fibromyalgia flareup in the subject.

It is still an object of the present invention to disclose the above mentioned method, additionally comprising steps of improving fibromyalgia symptoms of the subject as measured by at least one pain severity scale, compared to an established baseline, control or placebo.

It is still an object of the present invention to disclose the above mentioned method, additionally comprising steps of measuring the improvement in the fibromyalgia symptoms of the subject by an improvement in the subject's score of at least one point on the at least one pain severity scale, as compared to an established baseline or to a placebo.

It is still an object of the present invention to disclose the above mentioned method, additionally comprising steps of selecting the pain severity scale from a group consisting of brief pain inventory, Sleep history questionnaire (PSQI), SF-36 Quality of life assessment, Fibromyalgia impact questionnaire (FIQ), Mini International Neuropsychiatric Interview, fibromyalgia syndrome Tenderness Assessment, Hamilton Depression Rating Scale (HDRS) and any combination thereof.

It is still an object of the present invention to disclose the above mentioned method, additionally comprising steps of selecting the fibromyalgia symptoms from the group comprising of widespread pain, chronic pain, cognitive difficulties, chronic muscle pain, muscle spasms, muscle tightness, fatigue, insomnia, stiffness, fibro fog, abdominal pain, bloating, nausea, constipation, diarrhea, headache, jaw and facial tenderness, anxiety, depression, numbness or tingling in the limbs, irritable bladder, feeling of swelling, painful menstruation, restless leg, dizziness, female or subject with established FMS confirmed by the 1990 ACR, and any combination thereof.

It is still an object of the present invention to disclose the above mentioned method, additionally comprising steps of selecting the fibromyalgia symptoms to be a sensitivity to at least one of the following: odors, noise, bright lights, medications, certain foods, and cold.

It is still an object of the present invention to disclose the above mentioned method, additionally comprising steps of providing an improvement in fibromyalgia symptoms of said subject measured by a score on the average pain severity item of the BPI scale of above 5.

It is still an object of the present invention to disclose the above mentioned method, additionally comprising steps of providing a reduction in BPI score from baseline to endpoint.

It is still an object of the present invention to disclose the above mentioned method, additionally comprising steps of providing a sustained response defined as a 30% reduction from baseline to endpoint in the BPI score with a 30% reduction from baseline at least 2 weeks prior to the last, and with at least a 20% reduction from baseline at every week in between.

It is still an object of the present invention to disclose the above mentioned method, additionally comprising steps of providing an improvement in pain severity as measured by the self-reported Brief Pain Inventory (short form) average pain severity score fibromyalgia impact questionnaire items.

It is still an object of the present invention to disclose the above mentioned method, wherein said pain severity score fibromyalgia impact questionnaire items comprising items which measure physical function, pain assessment, fatigue and distress.

It is still an object of the present invention to disclose the above mentioned method, additionally comprising steps of providing improvement in measures selected from the group consisting of: quality of life, quality of sleep, disability, depression, anxiety and the patient global impression of change and the fibromyalgia severity score.

It is another object of the present invention to provide a use of a composition comprising tetrahydrocannabinol (THC) or a derivative thereof, or cannabidiol (CBD) or a derivative thereof, or a combination thereof, in the manufacture of a medicament to treat fibromyalgia syndrome.

It is yet another object of the present invention to provide a composition comprising a therapeutically effective amount of cannabis formulation for use in relieving a subject suffering from fibromyalgia syndrome symptoms, wherein the cannabis formulation contains synthetic components of cannabis.

It is also an object of the present invention to provide the aforementioned composition, wherein the synthetic components are synthetically produced tetrahydrocannabinol (THC) or a derivative thereof, or cannabidiol (CBD) or a derivative thereof, or a combination thereof.

It is also an object of the present invention to provide the aforementioned composition, further comprising a ratio of the THC, or a derivative thereof, to the CBD, or a derivative thereof, of 4:1 or 5:1, respectively.

It is also an object of the present invention to provide the aforementioned composition, wherein the composition is formulated for administration of between about 10 mg THC and about 160 mg THC per day, preferably between about 10 mg THC and about 100 mg THC per day.

It is also an object of the present invention to provide the aforementioned composition, wherein the composition is formulated for administration of between about 2 mg CBD and about 40 mg CBD per day, preferably between about 2 mg CBD and about 20 mg CBD per day.

It is another object of the present invention to disclose a method for preventing the risk for the onset of a fibromyalgia flareup, wherein the method comprises steps of: formulating a composition comprising tetrahydrocannabinol (THC) or a derivative thereof, or cannabidiol (CBD) or a derivative thereof, or a combination thereof; and administering the composition to a subject in risk for suffering from a fibromyalgia flareup according to a predetermined protocol.

It is still an object of the present invention to disclose the aforementioned method, wherein the step of formulating further comprises formulating the composition to comprise a ratio of the THC, or a derivative thereof, to the CBD, or a derivative thereof, of 4:1 or 5:1, respectively.

It is still an object of the present invention to disclose the aforementioned method, additionally comprising steps of administering the THC or a derivative thereof in a dosage of between about 10 mg THC and about 160 mg THC per day, preferably between about 10 mg THC and about 100 mg THC per day.

It is lastly an object of the present invention to disclose the aforementioned method, additionally comprising steps of administering the CBD or a derivative thereof in a dosage of between about 2 mg CBD and about 40 mg CBD per day, preferably between about 2 mg and about 20 mg CBD per day.

### DETAILED DESCRIPTION OF THE PREFERRED EMBODIMENTS

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not unnecessarily obscured.

The essence of the present invention is to provide a composition for treating fibromyalgia comprising cannabis extract, which may further contain tetrahydrocannabinol (THC), cannabidiol (CBD), or a combination thereof, or a synthetic cannabis composition, or a combination thereof.

Fibromyalgia patients frequently self-report using cannabis therapeutically to treat symptoms of the disease. To date however, there is still a need for clinical trials assessing the use of cannabinoids to treat the disease.

In a main embodiment, the present invention provides a composition comprising a therapeutically effective amount of Tetrahydrocannabinol (THC) or a derivative thereof, or Cannabidiol (CBD) or a derivative thereof, or a combination thereof for use in relieving a subject suffering from fibromyalgia syndrome symptoms. It is emphasised that the composition of the present invention is useful for treating chronic pain conditions such as fibromyalgia.

In a further embodiment, the composition of the present invention, comprising a therapeutically effective amount of Tetrahydrocannabinol (THC) or a derivative thereof, or Cannabidiol (CBD) or a derivative thereof, or a combination thereof is useful for treating chronic pain.

Without wishing to be bound by theory, it is herein acknowledged that the principal active component in the complex mixture of cannabinoids present in the marijuana plant is D9-tetrahydrocannabinol (THC), which acts primarily as an agonist at the CB1 cannabinoid receptor. This receptor is found at high concentrations in the brain, including the basal ganglia and cerebellar regions, and also in the hippocampus and hypothalamus. THC has been shown to inhibit the release of a wide spectrum of neurotransmitters including L-glutamate, GABA, norepinephrine, dopamine, serotonin (5-HT), and acetylcholine (Schlicker and Kathmann, 2001).

It is further acknowledged that marijuana contains other compounds of interest, including cannabidiol (CBD), a constituent of marijuana that is not a CB1 or CB2 receptor agonist (Burstein and Zurier, 2009; Scuderi et al., 2009). An early safety study reported that CBD was tolerated when administered to humans for 30 days (Cunha et al., 1980).

It is further within the scope that cannabinoids also modulate GABAergic transmission and the release of cholecystokinin (CCK), a peptide that may contribute to both anxiolytic and anxiogenic effects of THC and endocannabinoids (Beinfeld and Connolly, 2001; Katona et al., 1999; Marsicano and Lutz, 1999; Onaivi et al., 1990). Furthermore, cannabinoids enhance the release of endogenous opioids, and these may be involved in the functional interplay between the endocannabinoid and the opioid system and the production of analgesic responses. It is hypothesized that the relationship between these two systems plays a role in antidepressant-like effects and in various addiction-related processes (Houser et al., 2000; Pugh et al., 1997; Zimmer et al., 2001). Studies in rodents suggest that cannabinoids and their interaction with endogenous opioids might also modulate anxiety (Pugh et al., 1997; Berrendero and Maldonado, 2002; Marin et al., 2003).

It is an object of the present invention to use pharmacological modulation of the endocannabinoid system in the treatment of fibromyalgia related symptoms.

The composition of the present invention mainly comprises of cannabis extract drops. Such cannabis drops essentially comprises the cannabinoid Tetrahydrocannabinol (THC). The cannabis oil of the composition of the present invention may also include cannabidiol (CBD) that is known to potentiate the activity of THC by increasing CB1 receptor density or through another CB1-related mechanism (Hayakawa et al., 2008). CBD may increase the efficiency of the composition as well as will enable using lower concentrations of THC. The cannabis drops may also be formulated out of synthetic cannabis components or a combination of synthetic and naturally extracted components of cannabis.

As used herein the term “about” denotes ±25% of the defined amount or measure or value.

The term “cannabis” refers hereinafter to a genus of flowering plants that includes three different species, Cannabis sativa, Cannabis indica and Cannabis ruderalis.

The term “cannabidiol (CBD)” refers hereinafter to one of at least 85 active cannabinoids identified in cannabis. Cannabidiol is a major phytocannabinoid, accounting for up to 40% of the plant's extract. CBD is considered to have a wider scope of medical applications than tetrahydrocannabinol (THC). Cannabidiol has a very low affinity for CB1 and CB2 receptors but acts as an indirect antagonist of their agonists. CBD may potentiate THC's effects by increasing CB1 receptor density or through another CB1-related mechanism. It is also an inverse agonist of CB2 receptors. CBD possesses antiproliferative, pro-apoptotic effects and inhibits cancer cell migration, adhesion and invasion.

The term “Tetrahydrocannabinol (THC)” refers hereinafter to the principal psychoactive constituent (or cannabinoid) of the cannabis plant. THC has a partial agonist activity at the cannabinoid receptor CB1, and the CB2 receptor and is known to increase cortisol levels. THC may refer to delta-9-tetrahydrocannabinol, delta-6-tetrahydrocannabinol and delta-1-tetrahydrocannabinol.

The term “cannabinoid receptor” refers hereinafter to a class of cell membrane receptors under the G protein-coupled receptor superfamily. There are currently two known subtypes of cannabinoid receptors, termed CB1 and CB2. The CB 1 receptor is expressed mainly in the brain, but also in the lungs, liver and kidneys. The CB2 receptor is expressed mainly in the immune system and in hematopoietic cells.

The term “Cannabinoid receptor type 1 (CB1)” refers hereinafter to a G protein-coupled cannabinoid receptor located primarily in the central and peripheral nervous system. It is activated by the endocannabinoid neurotransmitters anandamide and 2-arachidonoyl glyceride (2-AG); by plant cannabinoids, such as the compound THC, an active ingredient of the psychoactive drug cannabis; and by synthetic analogues of THC.

The term “Cannabinoid receptor type 2 (CB2)” refers hereinafter to a G protein-coupled receptor from the cannabinoid receptor family that in humans is encoded by the CNR2 gene. It is closely related to the cannabinoid receptor type 1, which is largely responsible for the efficacy of endocannabinoid-mediated presynaptic-inhibition, the psychoactive properties of Tetrahydrocannabinol, the active agent in marijuana, and other phytocannabinoids (natural cannabinoids). The principal endogenous ligand for the CB2 receptor is 2-arachidonoylglycerol (2-AG).

The term “fibromyalgia” as used herein refers to a disorder characterized by widespread musculoskeletal pain accompanied by fatigue, sleep, memory and mood issues. It is acknowledged that women are more likely to develop fibromyalgia than men. It is noted that people who have fibromyalgia sometimes also have tension headaches, temporomandibular joint (TMJ) disorders, irritable bowel syndrome, anxiety and depression. It is within the scope that the term “fibromyalgia” further refers to chronic pain conditions and symptoms.

The term “chronic pain” as used herein refers a pain that lasts a long time. It is acknowledged that In medicine, the distinction between acute and chronic pain is sometimes determined by an arbitrary interval of time since onset; the two most commonly used markers being 3 months and 6 months since onset, though some theorists and researchers have placed the transition from acute to chronic pain at 12 months. Others apply acute to pain that lasts less than 30 days, chronic to pain of more than six months duration, and subacute to pain that lasts from one to six months.[3] A popular alternative definition of chronic pain, involving no arbitrarily fixed duration, is “pain that extends beyond the expected period of healing”. Epidemiological studies have found that 10.1% to 55.2% of people in various countries have chronic pain.

The term “sustained release dosage form” refers hereinafter to the release of a drug at a predetermined rate in order to maintain a constant drug concentration for a specific period of time with minimum side effects. This can be achieved through a variety of formulations, including liposomes and drug-polymer conjugates. Sustained release's definition is more akin to a “controlled release” rather than “sustained”.

The term “fibromyalgia severity scale” refers hereinafter to rating and scales that are commonly used as measures of symptom severity, treatment response and the efficacy of treatments in treatment studies of patients which exhibit fibromyalgia syndrome symptoms. The fibromyalgia severity scale may be selected from a few commonly used scales, which may include in a non-limiting example, pain severity scale, Brief pain inventory, Sleep history questionnaire (PSQI), SF-36 Quality of life assessment, Fibromyalgia impact questionnaire (FIQ), Mini International Neuropsychiatric Interview, fibromyalgia syndrome Tenderness Assessment, and Hamilton Depression Rating Scale (HDRS).

The term “established baseline” or “control” or “placebo” refers hereinafter to the results obtained by a subject or group of subjects that was not administered or otherwise exposed to the composition of the present invention and used as a control for the clinical study. Specifically, the term “placebo” refers to a substance containing no medication and prescribed or given to reinforce a patient's expectation to get well. According to further aspects, a placebo is a simulated or otherwise medically ineffectual treatment for a disease or other medical condition intended to deceive the recipient. Sometimes patients given a placebo treatment will have a perceived or actual improvement in a medical condition, a phenomenon commonly called the placebo effect. It is within the scope that in medical research, placebos are given as control treatments and depend on the use of measured suggestion. Examples of common placebos include inert tablets, vehicle infusions, sham surgery, and other procedures based on false information. The term “established baseline” refers hereinafter to data collected at the beginning of a clinical study for all participants and for each arm or comparison group. These data include demographics, such as age and gender, and study-specific measures (for example, systolic blood pressure, prior antidepressant treatment etc.).

The term “extract” refers hereinafter to any extract deriving from a plant, or fragment or fraction thereof.

Thus it is according to one embodiment of the present invention to provide a composition comprising tetrahydrocannabinol (THC) or a derivative thereof, or cannabidiol (CBD) or a derivative thereof, or a combination thereof, for use in relieving a subject suffering from fibromyalgia syndrome symptoms.

It is further within the scope of the present invention to disclose the composition as defined above, wherein the THC or a derivative thereof is selected from the group consisting of THC, THCV, THCA and any combination thereof.

It is further within the scope of the present invention to disclose the composition as defined in any of the above, wherein the THC or a derivative thereof is selected from the group consisting of natural THC or a derivative thereof produced in the body of humans and animals, THC or a derivative thereof extracted from plants, synthetic THC or a derivative thereof, and any combination thereof.

It is further within the scope of the present invention to disclose the composition as defined in any of the above, wherein the THC or a derivative thereof is extracted from cannabis; the cannabis is selected from the group consisting of: Cannabis sativa, Cannabis indica, Cannabis ruderalis, and any combination thereof.

It is further within the scope of the present invention to disclose the composition as defined in any of the above, wherein the composition additionally comprising cannabidiol (CBD) or a derivative thereof.

It is further within the scope of the present invention to disclose the composition as defined in any of the above, wherein the cannabidiol (CBD) or a derivative thereof is selected from the group consisting of CBD, CBDV, CBDA and any combination thereof.

It is further within the scope of the present invention to disclose the composition as defined in any of the above, wherein the CBD or a derivative thereof is selected from the group consisting of natural CBD or a derivative thereof produced in the body of humans and animals, CBD or a derivative thereof extracted from plants, synthetic CBD or a derivative thereof, and any combination thereof.

It is further within the scope of the present invention to disclose the composition as defined in any of the above, wherein the CBD or a derivative thereof is extracted from cannabis; the cannabis is selected from a group consisting of: Cannabis sativa, Cannabis indica, Cannabis ruderalis, and any combination thereof.

It is well within the scope of the present invention to disclose a cannabis formulation for the treatment of fibromyalgia syndrome symptoms, containing only synthetic cannabis components, or a combination of synthetic cannabis components with naturally extracted cannabis components.

It is further within the scope of the present invention to disclose a composition comprising a therapeutically effective amount of Tetrahydrocannabinol (THC) or a derivative thereof, or Cannabidiol (CBD) or a derivative thereof, or a combination thereof for use in relieving a subject suffering from fibromyalgia syndrome symptoms.

It is further within the scope of the present invention to disclose the composition as defined in any of the above, further comprising a ratio of said THC, or a derivative thereof, to said CBD, or a derivative thereof of 4:1 or 5:1, respectively.

It is further within the scope of the present invention to disclose the composition as defined in any of the above, wherein the composition is formulated for administration of between about 10 mg THC and about 160 mg THC per day, preferably between about 10 mg and about 100 mg THC per day, more preferably between about 10 mg and about 20 mg THC per day.

It is further within the scope of the present invention to disclose the composition as defined in any of the above, wherein said composition is formulated for administration of between about 5 mg THC and about 20 mg THC per dosage unit, preferably about 10 mg THC per dosage unit.

It is further within the scope of the present invention to disclose the composition as defined in any of the above, wherein the composition is formulated for administration of between about 2 mg CBD and about 40 mg CBD per day, preferably between about 2 mg and about 20 mg CBD per day, more preferably between about 2 mg and about 4 mg CBD per day.

It is further within the scope of the present invention to disclose the composition as defined in any of the above, wherein the composition is formulated for administration of between about 1 mg CBD and about 5 mg CBD per dosage unit, preferably about 2 mg CBD per dosage unit.

It is further within the scope of the present invention to disclose the composition as defined in any of the above, wherein the composition is formulated for administration of about 1 to about 10 times per day.

In order to understand the invention and to see how it may be implemented in practice, a plurality of preferred embodiments will now be described, by way of non-limiting example only, with reference to the following examples.

### EXAMPLE 1

Organic, mental and functional aspects of the fibromyalgia syndrome are assessed in patients treated with cannabis drops compared to placebo. The protocol-defined primary outcome measure is pain severity as measured by the self-reported Brief Pain Inventory (BPI) (short form) average pain severity score fibromyalgia impact questionnaire (which measures physical function, pain assessment, fatigue and distress).

Secondary endpoints include validated parameters that measure quality of life, quality of sleep, disability, depression and anxiety and the patient global impression of change and the fibromyalgia severity score. In addition changes of concurrent medications are recorded.

Sample Size: The study will include eighty patients.

Maximal Study Duration time: up to 7 visits in up to 23 weeks as follows:
Screening phase—up to 3 weeks, 1 visit
Treatment phase—16 weeks, 4 visits
Follow-up phase—4 weeks, 2 visits

Study Design: The study will be randomized and double-blinded, and will compare addition of cannabis drops versus placebo to female patients with fibromyalgia. Female patients with primary fibromyalgia syndrome will be recruited after obtaining their signed informed consent. The patients will be randomized to receive cannabis drops on an escalating scale or a placebo. Rescue therapy will be provided by paracetamol and/or dypirone drops in an unblinded manner. The duration of the study will be 6 weeks. There will be 4 biweekly office visits throughout these 6 weeks preceded by a screening visit. The study drug will be provided as an “add on” format.

The patients will start with the following drop dosage; every day of the first week each patient will take 5 drops, increments by five drops will be allowed by weekly intervals. The maximal dose will be no more than up to 20 drops twice daily. Dosage will be given at a 4:1 ratio between THC and CBD, wherein THC ranges from about 20 mg and about 160 mg per day, and CBD ranges from about 5 mg and 40 mg per day.

Inclusion Criteria: individuals eligible to be enrolled into this protocol are participants who:
1. Women with established fibromyalgia syndrome confirmed by the 1990 ACR classification criteria who signed an informed consent form.
2. Women 18-75 years old.
3. The score on the average pain severity item of the BPI is >5 at randomization.
4. Women of fertile age will need to use birth control measures.

Exclusion Criteria: individuals not eligible to be enrolled into this protocol are those who:
1. Confirmed pregnancy
2. Breast feeding patients
3. Patients with active coronary artery disease with documented myocardial ischemia proven by coronary angiography, thallium scan or exercise stress test.
4. Patients with congestive heart failure
5. Patients with coexistent neoplastic conditions (not including basal cell carcinoma)
6. Patients with coexistent t rheumatic/inflammatory conditions
7. Patients with active gastrointestinal bleeding
8. Patients with renal failure
9. Patients with comorbid conditions causing significant disability
10. Patients with uncontrolled hypertension.
11. Patients with significant psychiatric conditions (excluding depression and anxiety disorders).
12. A high level of suicidality (a score above 3 in the relevant Hamilton score).
13. Patients with current or past history of substance abuse.
14. Patients who are drivers of public transportation or heavy vehicles

### Experimental Design:

All women will be assessed by several rheumatic and psychiatric scales as following:

Brief pain inventory—Pain severity as measured by the self-reported BPI (short form) average pain severity score, which assesses average pain severity during the past 24 hours (0-10 scale, where 0=no pain and 10=pain as bad as you can imagine). A sustained response will be defined as a 30% reduction from baseline to endpoint in the BPI with a 30% reduction from baseline at least 2 weeks prior to the last, and with at least a 20% reduction from baseline at every week in between. The primary outcome variable will be reduction in BPI from baseline to endpoint.

Sleep history questionnaire (PSQI)—PSQI is a validated sleeping scoring that measures sleep quality, impact on function and well being. A final score result below 5 reflects poor quality of sleep.

SF-36 Quality of life assessment—A Hebrew translated and validated version of the SF-36 scale measured quality of life. The SF-36 contains eight subscales: physical functioning (PF), social functioning (SF), role limitations attributable to physical and emotional problems, mental health, vitality, bodily pain, and general health.

Fibromyalgia impact questionnaire (FIQ)—This scale commonly performed in patients with FIBROMYALGIA SYNDROME is regarded to be a reliable and valid variable. The FIQ is less affected by temporary alterations and fluctuations of the disease severity.

Mini International Neuropsychiatric Interview this tools provides rapid assessment for comorbid psychiatric conditions.

Fibromyalgia syndrome Tenderness Assessment—Tenderness assessment was performed manually in all subjects by a senior rheumatologist; a count of 18 tender points (TP) will be performed by thumb palpation as specified in the ACR 1990 classification criteria.

Hamilton Depression Rating Scale (HDRS)—Major depressive disorder severity was determined by using the Hamilton Depression Rating Scale (HDRS). The HDRS is a 17-item scale that measures the presence and severity of depression. The HDRS is a reliable gauge of the degree of symptom severity in depressed patients.

All adverse events will be recorded at every office visit. Every serious adverse events will recorded within 24 hours from occurrence and the local ethics committee will be notified immediately.

BMI—weight and height will be measured at the beginning and end of the study.

In addition a urine toxic screen will be taken every visit, the patients will be blinded to the results of this tests throughout the study.

Statistical analysis: Prospective results are detailed for all data.

Data analysis will be two folded: group comparisons and correlations. Differences between the two groups of patients for continuous variables will be analyzed using. For categorical variables, group comparisons will be performed with the chi square test. Correlations between TP and other dependent variables will be calculated using Pearson correlations. Scores measured at several time points will also be analyzed by repeated measures ANOVA, absolutely and as percentage of baseline values.

### EXAMPLE 2

### A Randomized Double-Blinded Study Comparing Treatment with the Cannabis Tablets of the Present Invention Versus Placebo in Female Patients with Fibromyalgia

### Abbreviations:

AE Adverse Event

BP Blood Pressure

CBD Cannabidiol

CRF Case Report Form

DCF Data Correction Form

ECG Electrocardiogram

EC Ethics Committee

FMS Fibromyalgia symptoms

GCP Good Clinical Practice

HCG Human Chorionic Gonadotropin

HIV Human Immunodeficiency Virus

HR Heart Rate

PI Principal Investigator

SAE Serious Adverse Event

THC Tetrahydrocannabinol

### 1. Protocol Synopsis

| | |
|---|---|
| Title | A randomized double-blinded study comparing treatment |
| | with Cannabis tablets versus placebo in female patients |
| | with fibromyalgia |
| Phase of | Phase II |
| Development | |
| Primary | To evaluate the safety, tolerability and efficacy of |
| Objectives | cannabis tablets (10 mg THC and 2 mg of CBD in female |
| | patients with fibromyalgia. |
| Primary | Pain severity as measured by the self-reported BPI (short |
| Outcome | form) average pain severity score fibromyalgia impact |
| Measures: | questionnaire (which measures physical function, pain |
| | assessment, fatigue and distress). |
| Secondary | 1. Quality of life, quality of sleep, disability, |
| Outcome | depression and anxiety and the patient global |
| Measures: | impression of change and the fibromyalgia |
| | severity score. |
| | 2. Changes of concurrent medications will be |
| | recorded. |
| Safety | 1. Collecting information on tolerability through self- |
| Outcome | reported reactions and experiences. |
| Measures: | 2. Collection of Adverse events and Serious Adverse |
| | Events. |
| | 3. Laboratory testing. |
| Study Design | This is a single center, prospective, double-blind, placebo- |
| | controlled, randomized study. |
| Study | The subjects are screened against eligibility criteria and in |
| Methods | order to confirm eligibility, subject will be tested for |
| | pregnancy (urine or blood), urine Cannabinoids (to |
| | exclude substance abuse). Patients will be consented and |
| | the following procedures will be performed: |
| | Subjects will be randomized to one of the study arms |
| | (treatment or placebo). |
| | Documentation of Medical history and medication |
| | consumption (present and past) |
| | BP, Heart Rate, Blood testing (chemistry, blood count). |
| | Fibromyalgia Tender Points counts. |
| | BMI calculation |
| | Subjects will be asked to fill the following questionnaires: |
| | Brief pain inventory, Sleep history questionnaire (PSQI), |
| | SF-36 Quality of life assessment, Fibromyalgia impact |
| | questionnaire (FIQ), Mini International Neuropsychiatric |
| | Interview, Hamilton Anxiety Rating Scale (HAM-A) and |
| | Hamilton Rating Scale for Depression (HRSD). |
| | Based on the urine testing and questionnaires, eligibility |
| | confirmation will be performed and the patient will be |
| | asked to swallow a tablet. 1 hour and 2 hours after tablet |
| | intake Blood tests for CBD and THC will be performed. |
| | Patients will be instructed to start home use of a single |
| | tablet at night time with a possible bidaily increment up |
| | to a maximum dosage of 10 tablets. The increments will |
| | be done at night time and when needed the other half |
| | dose will be taken every morning. |
| | On the follow-up visits (including final visit) the |
| | following will be performed: |
| | Documentation medication consumption. |
| | Urine testing for pregnancy and Cannabinoids. |
| | BP, Heart Rate, Blood testing (chemistry, blood count). |
| | Subjects will be asked to fill the following questionnaires: |
| | Brief pain inventory, Sleep history questionnaire (PSQI), |
| | SF-36 Quality of life assessment, Fibromyalgia impact |
| | questionnaire (FIQ), Mini International Neuropsychiatric |
| | Interview, Hamilton Anxiety Rating Scale (HAM-A) and |
| | Hamilton Rating Scale for Depression (HRSD). |
| | At the final visit, the un-used tablets will be collected. |
| | In case of an Adverse Event, patients will be asked to |
| | contact the clinic immediately. Rescue therapy will be |
| | provided by paracetamol and/or dypirone drops in an |
| | unblinded manner |
| Number of | Single Site |
| Sites | |
| Maximal | 4 biweekly visits in 6 weeks as follows: |
| Study | Screening visit—Week 0 |
| Duration | Follow-up visit—Week 2 and Week 4 |
| time: | Final visit—week 6 |
| | Flexibility of ±2 days will be permitted |
| Sample Size | 80 female subjects |
| Inclusion | 1. Female with established FMS confirmed by the |
| Criteria | 1990 ACR |
| | 2. 18-75 years old |
| | 3. The score on the average pain severity item of the |
| | BPI is ≥5 at randomization |
| | 4. A female subject is eligible to participate if she is |
| | of non-childbearing potential (postmenopausal or |
| | pre-menopausal females with a documented tubal |
| | ligation or hysterectomy). Females on hormone |
| | replacement therapy (HRT) and whose |
| | menopausal status is in doubt will be required to |
| | use one of the contraception methods in the |
| | protocol. |
| | 5. Willing to sign an Informed Consent |
| | 6. Agree not to participate in any other interventional |
| | clinical trials during the study |
| | 7. Agree to follow study instructions meticulously |
| Exclusion | 1. Confirmed pregnancy |
| Criteria | 2. Breast feeding patients |
| | 3. Patients with active coronary artery disease with |
| | documented myocardial ischemia proven by |
| | coronary angiography, thallium scan or exercise |
| | stress test |
| | 4. Patients with congestive heart failure |
| | 5. Patients with coexistent neoplastic conditions (not |
| | including basal cell carcinoma) |
| | 6. Patients with coexistent t rheumatic/inflammatory |
| | conditions |
| | 7. Patients with active gastrointestinal bleeding |
| | 8. Patients with renal failure |
| | 9. Patients with comorbid conditions causing |
| | significant disability |
| | 10. Patients with uncontrolled hypertension. |
| | 11. Patients with significant psychiatric conditions |
| | (excluding depression and anxiety disorders). |
| | 12. Usage of anti-epileptic drugs |
| | 13. A high level of suicidality (a score above 3 in the |
| | relevant Hamilton score). |
| | 14. Patients who are drivers of public transportation or |
| | heavy vehicles. |
| | 15. A history of drug or alcohol abuse, or a history of |
| | regular alcohol consumption within 6 months of |
| | the study defined as an average weekly intake of |
| | >14 drinks for males or >7 drinks for females. One |
| | drink is equivalent to 12 gram of alcohol: 12 |
| | ounces (360 mililiter [mL]) of beer, 5 ounces (150 |
| | mL) of wine or 1.5 ounces (45 mL) of 80 proof |
| | distilled spirits. |
| | 16. The subject has participated in a clinical trial and |
| | has received an investigational product within the |
| | following time period prior to the first dosing day |
| | in the current study: 30 days, 5 half-lives |
| | (whichever is longer). |
| | 17. Have any current problem or a history of |
| | substance abuse which, in the opinion of the |
| | investigator, might interfere with participation in |
| | the protocol. |
| | 18. Have used marijuana within a month of starting |
| | the study. |

### Fibromyalgia Assessment Tools

The following questionnaires are used in the study. They are validated and commonly used.

Brief pain inventory—The Brief Pain Inventory (BPI) is a self-administered questionnaire developed to assess pain and the impact of pain. It was developed for use in cancer pain, but has also been used in other chronic pain conditions.

Sleep history questionnaire (PSQI)—PSQI is a validated sleeping scoring that measures sleep quality, impact on function and wellbeing. A final score result below 5 reflects poor quality of sleep.

SF-36 Quality of life assessment—A Hebrew translated and validated version of the SF-36 scale measured quality of life. The SF-36 contains eight subscales: physical functioning (PF), social functioning (SF), role limitations attributable to physical and emotional problems, mental health, vitality, bodily pain, and general health. Each scale is scored on a VAS (0 to 100) with a high score indicating better health and less body pain. The completion of this study was conducted with the aid of a senior psychiatrist.

Fibromyalgia impact questionnaire (FIQ)—This scale commonly performed in patients with FMS is regarded to be a reliable and valid variable. The FIQ is less affected by temporary alterations and fluctuations of the disease severity.

Mini International Neuropsychiatric Interview this tools provides rapid assessment for comorbid psychiatric conditions.

FMS Tenderness Assessment—Tenderness assessment will be performed manually in all subjects by a senior rheumatologist; a count of 18 tender points (TP) will be performed by thumb palpation as specified in the ACR 1990 classification criteria. The subject was asked to say “yes” when the sensation altered from pressure to definite pain. Patients will be considered to have FMS if he complied with the ACR criteria defining the FMS e.g., having widespread musculoskeletal pain with excess tenderness in at least 11 of 18 the predefined anatomic sites.

Hamilton Anxiety Rating Scale (HAM-A) and Hamilton Rating Scale for Depression (HRSD). These two scales are widely used to gauge the severity of anxiety and depression. The HAM-A consists of 14 items designed to assess the severity of a patient's anxiety. Each of the 14 items contains a number of symptoms, and each group of symptoms is rated on a scale of zero to four, with four being the most severe. The HDRS is a 17-item scale that measures the presence and severity of depression. The HDRS is a reliable gauge of the degree of symptom severity in depressed patients.

1990 ACR—The American College of Rheumatology (ACR) classification criteria, developed in 1990, helped galvanize research on FMS. The criteria required the presence of widespread pain in combination with 11 or more of 18 specific tender point sites. Widespread pain was defined as “3 out of 4 quadrant” pain, including left- and right-sided and upper- and lower-segment pain, and axial pain.

### 3. Rationale

This study investigates the safety, tolerability and efficacy of cannabis tablets (10 mg THC and 2 mg of CBD) in female patients with fibromyalgia. The evaluation tools of the Fibromyalgia symptoms are commonly used and validated in multiple studies, thus, will allow investigating if the cannabis tablet is an efficient treatment.

### 4. Objectives

To evaluate the safety, tolerability and efficacy of cannabis tablets (10 mg THC and 2 mg of CBD) in female patients with fibromyalgia.

### 5. Risks and Benefits

Fibromyalgia symptoms, including pain, fatigue, sleep deprivation, and mood instability or depression, can be effectively treated with the use Cannabinoids from cannabis (Cannabis sativa) cannabinoids, mimic the body's own naturally produced endocannabinoids. The cannabinoids in cannabis bind to the same endocannabinoid receptors that are responsible for regulating body systems including pain, appetite, mood and memory.

There is an extensive literature on the risks of habitual marijuana use in humans, and a sizeable but considerably smaller literature on the acute effects of marijuana, including adverse events.

Most research examining risks of marijuana examine smoked marijuana, as vaporization is a relatively recent form of marijuana consumption. Thus most reported risks associated with ingesting or inhaling marijuana relate to its psychoactive effects, though marijuana can also produce acute effects on the cardiovascular system and continued use can produce effects on the pulmonary system.

In this study, the investigational product is in a tablet form and it is provided in escalating dose to prevent adverse events and undesired affects related to cannabis.

The controlled dosage of CBD and THC along with the strict patient monitoring, limit the risk to the participant.

### 6. Study Design

This is a single center, prospective, double-blind, placebo-controlled, randomized study.

TABLE 1
STUDY PROCEDURES PER VISIT

| | Visit number | | | |
|---|---|---|---|---|
| | 1 | 2 | 3 | 4 |
| | Time of Visit | | | |
| | Week | Week | week | week |
| | 1 | 2 | 4 | 6 |
| Information & Informed consent | x | | | |
| urine Cannabinoids & Pregnancy testing | x | x | x | x |
| Epidemiological & Demographic data | x | x | x | x |
| Medication Consumption | x | x | x | x |
| BP, Heart Rate | x | x | x | x |
| Blood testing (chemistry, blood count) | x | x | x | x |
| Randomization | x | | | |
| BMI | x | | | x |
| Patient global impression of change | | x | x | x |
| Brief pain inventory | x | x | x | x |
| Tender point count | x | | | |
| SF-36 | x | | | x |
| Hamilton depression score | x | | | x |
| Fibromyalgia impact questionnaire | x | | | X |
| CBD and THC blood testing | x * | | | |
| Adverse events | x | x | x | x |
| Re-confirmation of patient eligibility | | | | |
| Dispense study drug | x | x | x | collect |

*—on the baseline visit, one hour and 2 hours after tablet intake.

### 7. Study Population

Study population: 80 female subjects with established FMS confirmed by the 1990 ACR are enrolled after meeting the following inclusion and exclusion criteria:

### 7.1 Inclusion Criteria

Subjects must satisfy inclusion criteria to be enrolled into the study:

Individuals eligible to be enrolled into this protocol are participants who:

Female with established FMS confirmed by the 1990 ACR

18-75 years old

The score on the average pain severity item of the BPI is ≥5 at randomization

A female subject is eligible to participate if she is of non-childbearing potential (postmenopausal or pre-menopausal females with a documented tubal ligation or hysterectomy). Females on hormone replacement therapy (HRT) and whose menopausal status is in doubt will be required to use one of the contraception methods in the protocol.

Willing to sign an Informed Consent

Agree not to participate in any other interventional clinical trials during the study

Agree to follow study instructions meticulously

### 7.2 Exclusion Criteria

1. Confirmed pregnancy
2. Breast feeding patients
3. Patients with active coronary artery disease with documented myocardial ischemia proven by coronary angiography, thallium scan or exercise stress test
4. Patients with congestive heart failure
5. Patients with coexistent neoplastic conditions (not including basal cell carcinoma)
6. Patients with coexistent t rheumatic/inflammatory conditions
7. Patients with active gastrointestinal bleeding
8. Patients with renal failure
9. Patients with comorbid conditions causing significant disability
10. Patients with uncontrolled hypertension.
11. Patients with significant psychiatric conditions (excluding depression and anxiety disorders).
12. Usage of anti-epileptic drugs
13. A high level of suicidality (a score above 3 in the relevant Hamilton score).
14. Patients who are drivers of public transportation or heavy vehicles.
15. A history of drug or alcohol abuse, or a history of regular alcohol consumption within 6 months of the study defined as an average weekly intake of >14 drinks for males or >7 drinks for females. One drink is equivalent to 12 gram of alcohol: 12 ounces (360 mililiter [mL]) of beer, 5 ounces (150 mL) of wine or 1.5 ounces (45 mL) of 80 proof distilled spirits.
16. The subject has participated in a clinical trial and has received an investigational product within the following time period prior to the first dosing day in the current study: 30 days, 5 half-lives (whichever is longer).
17. Have any current problem or a history of substance abuse which, in the opinion of the investigator, might interfere with participation in the protocol.

### 8. Study Procedures

The subjects are screened against eligibility criteria and in order to confirm eligibility, subject will be tested for pregnancy, urine Cannabinoids (to exclude substance abuse). Patients will be consented and the following procedures will be performed:
Subjects will be randomized to one of the study arms (treatment or placebo).
Documentation of Medical history and medication consumption (present and past)
BP, Heart Rate, Blood testing (chemistry, blood count).
Fibromyalgia Tender Points counts.
BMI calculation
Subjects will be asked to fill the following questionnaires: Brief pain inventory, Sleep history questionnaire (PSQI), SF-36 Quality of life assessment, Fibromyalgia impact questionnaire (FIQ), Mini International Neuropsychiatric Interview, Hamilton Anxiety Rating Scale (HAM-A) and Hamilton Rating Scale for Depression (HRSD).

Based on the urine testing and questionnaires, eligibility confirmation will be performed and the patient will be asked to swallow a tablet. 1 hour and 2 hours after tablet intake Blood tests for CBD and THC will be performed.

Patients are instructed to start home use of a single tablet at night time with a possible bidaily increment up to a maximum dosage of 10 tablets. The increments are done at night time and when needed the other half dose is taken every morning.

On the follow-up visits (including final visit) the following is performed:
Documentation medication consumption.
Urine testing for pregnancy and Cannabinoids.
BP, Heart Rate, Blood testing (chemistry, blood count).
Subjects will be asked to fill the following questionnaires: Brief pain inventory, Sleep history questionnaire (PSQI), SF-36 Quality of life assessment, Fibromyalgia impact questionnaire (FIQ), Mini International Neuropsychiatric Interview, Hamilton Anxiety Rating Scale (HAM-A) and Hamilton Rating Scale for Depression (HRSD).

At the final visit, the un-used tablets are collected.

In case of an Adverse Event, patients will be asked to contact the clinic immediately. Rescue therapy will be provided by paracetamol and/or dypirone drops in an unblinded manner.

### 8.1 Unscheduled Visits

An unscheduled visit is a visit unrelated to the study visits. It should be distinguished from an “Out of Window” visit that was performed outside the ±2 day visit window allowed per protocol, but was planned. In case of an “Out of Window” visit, the data collected at the visit is recorded in the closest scheduled visit CRF pages.

### 9. Treatment Principles

### 9.1 Method of Subject Identification

Subjects signing the informed consent are assigned a unique screening identifying number (3 digits number) and additional unique randomization number.

9.2 Removal of Subjects from Therapy or Assessment

All subjects are free to withdraw from participation in the study at any time, for any reason, without prejudice. The investigator may terminate a subject from the study at any time for lack of therapeutic effect that is intolerable to the subject or otherwise considered unacceptable, for intolerable or unacceptable AE's, inter-current illness, noncompliance with study procedures, or in the investigator's opinion to protect the subject's best interest.

All subjects prematurely discontinuing the study must be seen for a final evaluation unless the subject withdrew consent. If a subject is withdrawn before completing the study, the date and reason for withdrawal are entered on the appropriate page of the case report form (CRF). If study withdrawal was due to an AE, the subject should be followed until resolution or until the principal investigator deems it to be no longer medically indicated.

The primary reason for a premature withdrawal will be selected from the following standard categories of early termination: Adverse Event (AEs): Clinical or laboratory events which occurred that in the medical judgment of the investigator for the best interest of the subject are grounds for discontinuation. This includes serious and non-serious AEs regardless of relation to the study product.
1. Death of the subject.
2. Withdrawal due to Pregnancy.
3. Withdrawal of Consent: The subject desired to withdraw from further participation in the study in the absence of a medical need to withdraw as determined by the investigator. If the subject gave a reason for this desire, this should be recorded in the CRF.
4. Protocol Violation: The subject's findings or conduct failed to meet the protocol entry criteria or failed to adhere to the protocol requirements (e.g. treatment noncompliance, failure to return for defined number of visits). The violation necessitated premature termination from the study.
5. Lost to Follow-Up: The subject stopped coming for visits and study personnel were unable to contact the patient.
   6. Other: The subject was terminated for a reason other than those listed above (e.g., termination of study by Principal Investigator). The actual reason should be entered into the CRF.

### 9.3 Concomitant Therapy

Medications, not allowed prior to the start of the study, are listed in the exclusion criteria.

In case of new medications or changes in dosing of existing medications, the drug, reason for use, dose and dosage regimen must be recorded in the Case Report Form.

Females must continue the use of contraceptives. These must be recorded in the Case Report Form.

For any concomitant therapy given as a treatment for a new condition or a worsening of an existing condition, the condition must be documented on the Adverse Event Form of the CRF.

### 9.4 Laboratory Tests

Blood samples for biochemistry, serology and haematology including and a urine sample for urinalysis and drugs screen are taken as defined in the protocol and as per PI decision.

Female subjects will provide a urine sample for a pregnancy test during the screening visit.

The Medical Center local laboratory test results will be transferred to the CRF. The lab report must be interpreted, signed and dated by the principal investigator or his/her designee; any clinically significant changes occurring during the study must be recorded on the AE Form of the CRF.

### 9.4.1 Chemistry:

Glucose, Urea, Creatinine, Sodium, Potassium, Chloride, Calcium, Phosphorus, Uric Acid, AST (SGOT), ALT (SGPT), Gamma GT, LDH, Alkaline Phosphatase, Total Bilirubin, Direct Bilirubin, Total Protein, Albumin, Total Cholesterol, HDL, LDL, Trig

### 9.4.2 Hematology:

Haemoglobin, Haematocrit, RBC, MCV, WBC, White differential blood cell count (neutrophils, lymphocytes, monocytes, eosinophils, basophils), Platelets count, MPV.

### 9.4.3 Urinalysis

A 20 ml midstream urine sample must be provided for urinalysis for Cannabinoids.

### 9.5 Vital Signs

Blood pressure and heart rate measurements are assessed either manually by a sphygmomanometer or by an automated blood pressure device.

Heart rate and blood pressure are obtained at specified time points after subject has been in a supine position for 5 minutes.

### 10. Investigational Product (MGC, Ointment)

The principal investigator must maintain an adequate record of the receipt, distribution and return or destruction of all MGC tubes using an appropriate accountability forms provided for the study by the Sponsor. These forms must be available for inspection at any time.

### 10.1 Packing and Labelling

All tablets are packed and labeled in compliance with the EUROPEAN COMMISSION, Good Manufacturing Practice, Annex 13 (Brussels, 3 Feb. 2010) and according to the Israeli Guidelines for Clinical Trials in Human Subjects, 2006 (section 11 from December 2008).

### 11. Statistical Methods

### 11.1 Sample Size Justification

A sustained response is defined as a 30% reduction from baseline to endpoint in the BPI with a 30% reduction from baseline at least 2 weeks prior to the last, and with at least a 20% reduction from baseline at every week in between. The primary outcome variable is the reduction in BPI from baseline to endpoint.

### 12. Adverse Events

### 12.1 Definitions

### 12.1.1 Adverse Event (AE):

Any untoward medical occurrence, unintended disease or injury, or untoward clinical signs (including abnormal laboratory findings) in subjects, users or other persons, whether or not related to the investigational medical device.

### 12.1.2 Serious Adverse Event (SAE):

Adverse event that
a) Led to death,
b) Led to serious deterioration in the health of the subject, that either resulted in
   1) A life-threatening illness or injury, or
   2) A permanent impairment of a body structure or a body function, or
   3) In-patient or prolonged hospitalization, or
   4) Medical or surgical intervention to prevent life-threatening illness or injury or permanent impairment to a body structure or a body function,
c) Led to fetal distress, fetal death or a congenital abnormality or birth defect
   Note: Planned hospitalization for a pre-existing condition, or a procedure required by the CIP, without serious deterioration in health, is not considered a serious adverse event.

### 12.1.3 Unexpected Adverse Event

An adverse event of which the nature or severity of which is not consistent with the applicable product information.

### 12.1.4 Life-Threatening

Any event in which the subject was at risk of death at the time of the event; it does not refer to an event which hypothetically might have caused death if it were more severe.

### 12.1.5 Adverse Event Severity

The investigator rates the severity of an adverse event as follows:
Grade 1—Mild AE
Grade 2—Moderate AE
Grade 3—Severe AE
Grade 4—Life threatening or disabling AE
Grade 5—Death related to AE

### 12.1.6 Adverse Event Attribution

Attribution definitions are in accordance with the NCI-CTEP guidelines for adverse events reporting.

An adverse event is considered associated with the use of the MGC ointment if the attribution is possible, probable or definite.

Adverse event attribution categories:

Unrelated—The AE is clearly not related to the use of the MGC ointment.

Unlikely—The AE is doubtfully related to the use of the MGC ointment.

Possible—The AE may be related to the use of the MGC ointment.

Probable—The AE is likely related to the use of the MGC ointment.

Definite—The AE is clearly related to the use of the MGC ointment.

### 12.1.7 Adverse Event Reporting

Adverse events or baseline signs and symptoms that occur during the screening period, between signing of the Informed Consent and actual MGC ointment treatment will be documented as part of the applicable medical history page(s) and adverse events pages of the CRF which will allow for designation of the event/symptom as a baseline event/symptom.

Adverse events occurring after the MGC ointment treatment will be reported on the adverse events CRF page and followed to satisfactory resolution. New adverse events will be recorded 1 week post end of MGC ointment active treatment visit.

SAEs should be reported by the investigator to the sponsor within 24 hours of their occurrence by telephone, facsimile (fax) or e-mail. The principal investigator must provide the minimal information: i.e. study number, subject's initials and date of birth, investigational product code number, period of administration, nature of the adverse event and the principal investigator's opinion of the attribution of a MGC to the SAE. This report of an SAE by telephone must always be confirmed by a written, more detailed report on the provided SAE reporting forms.

The Investigator shall report immediately, within 48 hours of actual knowledge of the event, to the chairman of the Institutional Ethics Committee, the following events:
death;
unexpected SAE when a connection between the event and use of the investigational product cannot be excluded;
any malfunction in an investigational product that can compromise the safety and efficacy of the investigational product.

The Investigator shall issue the report on the provided SAE reporting form.

New SAEs will be reported up to 2 weeks following the end of active treatment visit.

Follow up of ongoing adverse events will continue until resolution or until the principal investigator deems them to be no longer medically indicated.

Mortality will be reported as an SAE. The date of death, cause of death of the subject in a clinical study, whether the event is expected or associated with the investigational agent and autopsy findings if applicable will be recorded in the CRF.

Pregnancies occurring during clinical study must be reported to Sponsor/EC within 24 hours using the provided reporting form. The outcome of the pregnancy must also be reported to Sponsor/EC.

Appropriate reporting of adverse events to the regulatory authorities will be performed. All serious adverse events that are unexpected and associated with the use of MGC to the principal investigator(s) will be reported.

Unexpected Serious Adverse Effect that somehow related to treatment with the investigational product occurring in sites in Israel, shall be report to the Ministry of Health, the Investigators and all the parties involved in the trial, according to the following timetable:
A. Cases of death or life-threatening events shall be reported within 7 days of the Sponsor's actual knowledge of the event.
B. All other events shall be reported within 15 days of the Sponsor's actual knowledge of the event.

It is the principal investigator's responsibility to report such adverse events to the Local Ethics Committee (EC) which has approved the protocol unless otherwise required and documented by the EC.

Each subject will be provided with a “study card” indicating the name of the MGC ointment and indication, the study number, the principal investigator's name, the site's name and a 24-hour emergency contact number.

### 13. Study Closure Considerations

Reasons for closure of the investigational site(s) or termination of the study by the sponsor may include:
1. Successful completion of the study at the center;
2. The required number of subjects for the study has been recruited;
3. Failure of the principal investigator to comply with the protocol or GCP guidelines;
4. Safety concerns;
5. Sufficient data suggesting lack of efficacy;
6. Inadequate recruitment of subjects by the principal investigator(s)

### 14. Ethics and Regulations

This study is conducted in accordance with the following guidelines and regulations:
Declaration of Helsinki 2008: Sixth revision, 59th Meeting, Seoul,
Public Health Regulations: Clinical Trials in Human Subjects, Israel Ministry of Health, 1980
Guidelines for Clinical Trials in Human Subjects, Israel Ministry of Health, 2014.

These standard and guidelines addresses good clinical practice for the design, conduct, recording and reporting of clinical investigations carried out in human subjects to assess the safety or efficacy for regulatory purposes.

Compliance with this standard provides public assurance that the rights, safety and well-being of study subjects are protected, consistent with the principles that have their origin in the Declaration of Helsinki, and that the clinical study data are credible.

### 15. Study Materials And Documentation

The principal investigator acknowledges that the study medications is an investigational and as such must be handled strictly in accordance with the protocol and the container label. Study medication must be verified upon receipt and retained in a safe, secure location and stored under the appropriate conditions as specified on delivery. Study medication should be dispensed under the supervision of the principal investigator's designee such as the medical center pharmacist (if applicable). All dispensing must be performed by the site recorded on the Investigator's Statement.

### 15.1 Subject Information and Informed Consent

The informed consent process will be conducted in accordance with “Good clinical practice”. Prior to entry in the study, the principal investigator or his/her designee must explain to potential subjects the study and the implications of participation. Subjects will be informed that their participation is voluntary and that they may withdraw consent to participate at any time. They will be informed that choosing not to participate will not impact on the care that the subject will receive for the treatment of his/her disease. Subjects will be told that alternative treatments are available if they refuse to take part and that such refusal will not prejudice future treatment. Finally, they will be told that their records may be accessed by competent authorities and CRO without violating the confidentiality of the subject, to the extent permitted by the applicable law(s) and/or regulations. By signing the Informed Consent Form (ICF) the subject is authorizing such access.

After this explanation and before entry to the study, written, dated and signed informed consent should be obtained from the subject or legally acceptable representative.

The subject will be given sufficient time to read the Informed Consent Form and to ask questions. After this explanation and before entry to the study, consent should be appropriately recorded by means of both the subject's dated signature and the principal investigator's designee who conducted the informed consent discussion. After having obtained the consent, a copy of the Informed Consent must be given to the subject.

The informed consent form will contain contact details for the subject in case of pertinent questions about subject rights and in case of research related injury.

In case the subject is unable to read, an impartial witness must attest the informed consent. Subjects who are unable to comprehend the information provided are unable to be enrolled.

### 15.2 Source Data & Source Documents

If data are inadvertently recorded directly into the CRF, there should be, at a minimum, an entry in the medical record that each of the assessments was done, by whom, and the date it was done.

The nature and location of all source documents will be identified to ensure that all sources of original data required to complete the CRF are known to the company and investigational staff and are accessible for verification by the monitor. If electronic records are maintained, the method of verification must be discussed between the investigational staff and the monitor.

Source documents should include the following information for each subject:
subject identification (name, date of birth, gender)
documentation that subject meets eligibility criteria, i.e., history, physical examination, and confirmation of diagnosis (to support inclusion and exclusion criteria)
participation in the study (including study number)
study discussed and informed consent forms
dates of visits
documentation that protocol specific procedures were performed
results of efficacy parameters, as required by the protocol
Investigational product start and end date
record of all adverse events and other safety parameters
concomitant medication
date of study completion and reason for early discontinuation, if applicable

Each laboratory test will be reviewed for clinical significance, signed and dated by the Principal Investigator's designees/Co-PI.

### 15.3 Case Report Form

All data relating to the study must be recorded on CRFs. These CRFs are to be completed in a timely fashion, with the exception of results of tests performed outside the investigator's office, so that they always reflect the latest observations on the subjects participating in the study.

The CRF will be compared with the source documents to ensure that there are no discrepancies between data. All entries, corrections and alterations are to be made by the principal investigator's designees.

### 15.4 Subject Identification Log

In order to permit easy identification of the individual subject during and after the study, the principal investigator is responsible for keeping an updated Subject Identification Log. The Subject Identification Log records the personal identification of all subjects that have signed the informed consent form. This document will be reviewed by the monitor for completeness. However, in order to ensure subject confidentiality, no copy will be made.

### 15.5 Archiving

The principal investigator shall maintain the study documents. Study document include those listed in “Good clinical practice” and any additional documents required by the applicable regulatory requirement(s). The principal investigator should take measures to prevent accidental or premature destruction of these documents.

Essential documents should be retained until at least 2 years after the last approval of a marketing application and until there are no pending or contemplated marketing applications, or at least until 2 years have elapsed since the formal discontinuation of clinical development of the investigational product. These documents should be retained for a longer period however if required by the applicable regulatory requirements.

If it becomes necessary for the appropriate Regulatory Authority to review any documentation relating to this study, the principal investigator must permit access to such reports.

### 15.6 Protocol Modification

Any protocol modification must be documented in writing. Any change in the research activity, except that necessary to remove an apparent immediate hazard to the patient, must be reviewed and approved by the local ethics committee before implementation and submitted to the regulatory authorities.

### 15.7 Privacy of Personal Data

The processing of personal data in pursuit of this study will be limited to those data that are reasonably necessary to investigate the efficacy, safety, quality and utility of the investigational product(s) used in this study.

These data will be processed with adequate precautions to ensure confidentiality.

It is ensured that the personal data are:
1. Collected for a specified and legitimate purpose
2. Processed fairly and lawfully
3. Accurate and up to date

Explicit consent for the processing of personal data will be obtained from the participating subject (or his/her legally acceptable representative) prior to any processing of personal data.

This confidentiality will be maintained throughout the complete data processing.

1.-87. (canceled)

88. A composition comprising a therapeutically effective amount of Tetrahydrocannabinol (THC) or a derivative thereof, or Cannabidiol (CBD) or a derivative thereof, or a combination thereof for use in relieving a subject suffering from fibromyalgia syndrome symptoms.

89. The composition of claim 88, wherein at least one of the following holds true:
a) said Tetrahydrocannabinol (THC) or a derivative thereof, or said Cannabidiol (CBD) or a derivative thereof is from cannabis extract;
b) said THC a derivative thereof is selected from the group consisting of THC, Tetrahydrocannabivarin (THCV), Tetrahydrocannabinolic acid (THCA) and any combination thereof;
c) said THC or a derivative thereof is selected from the group consisting of natural THC or a derivative thereof produced in the body of humans and animals, THC or a derivative thereof extracted from plants, synthetic THC or a derivative thereof, and any combination thereof;
d) said THC or a derivative thereof is extracted from cannabis; said cannabis is selected from the group comprising of: Cannabis sativa, Cannabis indica, Cannabis ruderalis, and any combination thereof;
e) said cannabidiol (CBD) or a derivative thereof is selected from the group consisting of CBD, cannabidivarin (CBDV), cannabidiolic acid (CBDA) and any combination thereof;
f) said CBD or a derivative thereof is selected from the group consisting of natural CBD or a derivative thereof produced in the body of humans and animals, CBD or a derivative thereof extracted from plants, synthetic CBD or a derivative thereof, and any combination thereof;
g) said CBD or a derivative thereof is extracted from cannabis; said cannabis is selected from a group consisting of: Cannabis sativa, Cannabis indica, Cannabis ruderalis, and any combination thereof;
h) the concentration of said THC is in the range of about 2% to about 85%; and
i) the concentration of said CBD is in the range of about 2% to about 85%.

90. The composition of claim 88, further comprising a ratio of said THC, or a derivative thereof, to said CBD, or a derivative thereof of 4:1 or 5:1, respectively.

91. The composition of claim 88, wherein said composition is formulated for administration of at least one of:
a) between about 10 mg THC and about 160 mg THC per day, preferably between about 10 mg and about 100 mg THC per day;
b) between about 5 mg THC and about 20 mg THC per dosage unit, preferably about 10 mg THC per dosage unit;
c) between about 2 mg CBD and about 40 mg CBD per day, preferably between about 2 mg and about 20 mg CBD per day;
d) between about 1 mg CBD and about 5 mg CBD per dosage unit, preferably about 2 mg CBD per dosage unit; and
e) about 1 to about 10 times per day.

92. The composition of claim 88, wherein at least one of the following holds true:
a) said composition is administered in a manner selected from the group consisting of: intranasal, transdermal, topical, intravenous, oral, and any combination thereof;
b) said composition is formulated in the form of drops;
c) said composition is formulated in a dosage form selected from the group consisting of oil, liquid, solid, gas, oral, pill, tablet, capsule, buccal, sub-lingual, orally-disintegrating, thin film, liquid solution, suspension, powder or liquid or solid crystals, pastes, inhalational, aerosol, inhaler, nebulizer, smoking, vaporizer, parenteral, intradermal, intramuscular, intraosseous, intraperitoneal, intravenous, subcutaneous, topical, cream, gel, liniment or balm, lotion, ointment, drops, skin patch, vaginal, suppository, pessary, rectal and any combination thereof; and
d) said composition is administered in combination with at least one therapeutic agent selected from the group comprising of dypirone, pregabalin, duloxetine, milnacipran, tricyclic antidepressants, amitriptyline, gabapentin, paracetamol, tramadol, codeine, growth hormone, sodium oxybate, cyclobenzaprine, tizanidine, pramipexole, ropinirole, quercetin and any combination thereof.

93. The composition according to claim 92, wherein said composition provides a synergistic effect with respect to treatment of fibromyalgia when administered in combination with said therapeutic agent, as compared to the effect provided when said composition and said therapeutic agent administered separately.

94. The composition of claim 88, wherein at least one of the following holds true:
a) said composition additionally comprises at least one carrier or excipient selected from the group consisting of diluents, antiadherents, binders, coatings, disintegrants, surfactants, dissolving agents, solubilising agents, bioadhesive agents, polysaccharides, polymers, copolymers, fast dissolving tablet (FDT) type excipient, bioavailability enhancing agent, Thin Film type excipient, PharmFilm type excipient, mucoadhesive type excipient, acidifying agents, probiotic agents, protective agents, antioxidants, effervescent excipient, dispersing agents flavours, colours, lubricants, glidants, sorbents, preservatives, sweeteners, and any combination thereof;
b) said composition further comprising flavoring agents, selected from the group consisting of sugar, sucrose, sorbitol, sucralose, saccharin sodium, sodium cyclamate, aspartame, neotame, acesulfame potassium, stevioside, sodium chloride, D-limonene, citric acid and any combination thereof;
c) said composition further comprising preservatives, selected from the group consisting of methylparabens, ethylparabens, propylparabens, butylparabens, sorbic acid, acetic acid, propionic acid, sulfites, nitrites, sodium sorbate, potassium sorbate, calcium sorbate, benzoic acid, sodium benzonate, potassium benzonate, calcium benzonate, sodium metabisulfite, propylene glycol, benzaldehyde, butylated hydroxytoluene, butylated hydroxyanisole, formaldehyde donors, essential oils, monoglyceride, phenol, mercury components and any combination thereof;
d) said composition is in a sustained release dosage form or in an immediate release dosage form; and
e) said composition is in a sustained release dosage form selected from the group comprising of liposomes, drug polymer conjugates, microencapsulation, controlled-release tablet coating, and any combination thereof.

95. The composition of claim 88, wherein said composition is used for preventing the onset of a flareup of fibromyalgia symptoms in said subject.

96. The composition of claim 88, wherein said composition provides an improvement in fibromyalgia symptoms of said subject as measured by at least one pain severity scale, compared to an established baseline, control or placebo.

97. The composition of claim 96, wherein at least one of the following holds true:
a) said improvement in fibromyalgia symptoms is measured by an improvement in the subject's score of at least one point on said at least one pain severity scale, as compared to an established baseline or to a placebo;
b) said at least one pain severity scale is selected from the group comprising of Brief pain inventory (BPI), Sleep history questionnaire (PSQI), SF-36 Quality of life assessment, Fibromyalgia impact questionnaire (FIQ), Mini International Neuropsychiatric Interview, Hamilton Anxiety Rating Scale (HAM-A) and Hamilton Rating Scale for Depression (HRSD), fibromyalgia syndrome Tenderness Assessment, Hamilton Depression Rating Scale (HORS) and any combination thereof;
c) said fibromyalgia symptoms are selected from the group comprising of widespread pain, chronic pain, cognitive difficulties, chronic muscle pain, muscle spasms, muscle tightness, fatigue, insomnia, stiffness, fibro fog, abdominal pain, bloating, nausea, constipation, diarrhea, headache, jaw and facial tenderness, anxiety, depression, numbness or tingling in the limbs, irritable bladder, feeling of swelling, painful menstruation, restless leg, dizziness, female or subject with established FMS confirmed by the 1990 ACR and any combination thereof; and
d) a sensitivity to at least one of the following: odors, noise, bright lights, medications, certain foods, and cold.

98. The composition of claim 97, wherein said composition provides at least one of:
a) an improvement in fibromyalgia symptoms of said subject measured by a score on the average pain severity item of the BPI scale of above 5;
b) a reduction in BPI score from baseline to endpoint; and
c) a sustained response defined as a 30% reduction from baseline to endpoint in the BPI score with a 30% reduction from baseline at least 2 weeks prior to the last, and with at least a 20% reduction from baseline at every week in between.

99. The composition of claim 88, wherein said composition provides at least one of:
a) an improvement in pain severity as measured by the self-reported Brief Pain Inventory (short form) average pain severity score fibromyalgia impact questionnaire items; and
b) improvement in measures selected from the group consisting of: quality of life, quality of sleep, disability, depression, anxiety and the patient global impression of change and the fibromyalgia severity score.

100. The composition of claim 99, Wherein said pain severity score fibromyalgia impact questionnaire items comprising items which measure physical function, pain assessment, fatigue and distress.

101. A method for treating a subject suffering from fibromyalgia syndrome comprising steps of administering to said subject a composition comprising a therapeutically effective amount of Tetrahydrocannabinol (THC) or a derivative thereof, or Cannabidiol (CBD) or a derivative thereof, or a combination thereof in a therapeutically effective dosage.

102. The method of claim 101, additionally comprising at least one step selected from the group consisting of:
a) formulating said composition to comprise a ratio of said THC, or a derivative thereof, to said CBD, or a derivative thereof, of 4:1 or 5:1, respectively;
b) administering said THC or a derivative thereof in a dosage unit of between about 5 mg THC and about 20 mg THC, preferably about 10 mg THC per dosage unit;
c) administering said CBD or a derivative thereof in a dosage of between about 2 mg CBD and about 40 mg CBD per day, preferably between about 2 mg and about 20 mg CBD per day;
d) administering said CBD or a derivative thereof in a dosage unit of between about 1 mg CBD and about 5 mg CBD, preferably about 2 mg CBD per dosage unit;
e) formulating said composition in a dosage form of drops;
f) preventing the onset of a fibromyalgia flareup in said subject;
g) improving fibromyalgia symptoms of said subject as measured by at least one pain severity scale, compared to an established baseline, control or placebo;
h) providing an improvement in fibromyalgia symptoms of said subject measured by a score on the average pain severity item of the BPI scale of above 5;
i) providing a reduction in BPI score from baseline to endpoint;
j) providing a sustained response defined as a 30% reduction from baseline to endpoint in the BPI score with a 30% reduction from baseline at least 2 weeks prior to the last, and with at least a 20% reduction from baseline at every week in between; and
k) providing an improvement in pain severity as measured by the self-reported Brief Pain Inventory (short form) average pain severity score fibromyalgia impact questionnaire items.

103. The method of claim 102, wherein at least one of the following folds true:
a) the method additionally comprising steps of performing blood tests for CBD and THC 1 hour and 2 hours after said dosage unit intake;
b) the method additionally comprising steps of selecting said fibromyalgia symptoms to be a sensitivity to at least one of the following: odors, noise, bright lights, medications, certain foods, and cold; and
c) said pain severity score fibromyalgia impact questionnaire items comprising items which measure physical function, pain assessment, fatigue and distress.

104. The method of claim 103, additionally comprising steps of providing improvement in measures selected from the group consisting of: quality of life, quality of sleep, disability, depression, anxiety and the patient global impression of change and the fibromyalgia severity score.

105. A composition comprising a therapeutically effective amount of cannabis formulation for use in relieving a subject suffering from fibromyalgia syndrome symptoms, wherein said cannabis formulation contains synthetic components of cannabis.

106. The composition of claim 105, wherein at least one of the following holds true:
a) said synthetic components are synthetically produced tetrahydrocannabinol (THC) or a derivative thereof, or cannabidiol (CBD) or a derivative thereof, or a combination thereof;
b) said composition comprising a ratio of said THC, or a derivative thereof, to said CBD, or a derivative thereof, of 4:1 or 5:1, respectively;
c) said composition is formulated for administration of between about 10 mg THC and about 160 mg THC per day, preferably between about 10 mg and about 100 mg THC per day; and
d) said composition is formulated for administration of about 2 mg CBD and about 40 mg CBD per day, preferably between about 2 mg CBD and about 20 mg CBD per day.

107. A method for preventing the risk for the onset of a fibromyalgia flareup, wherein said method comprises steps of:
a) formulating a composition comprising tetrahydrocannabinol (THC) or a derivative thereof, or cannabidiol (CBD) or a derivative thereof, or a combination thereof; and
b) administering said composition to a subject in risk for suffering from a fibromyalgia flareup according to a predetermined protocol.

108. The method of claim 107, wherein at least one of the following holds true:
a) said step of formulating further comprises formulating said composition comprises a ratio of said THC, or a derivative thereof, to said CBD, or a derivative thereof, of 4:1 or 5:1, respectively;
b) said method additionally comprising steps of administering said THC or a derivative thereof in a dosage of about 10 mg THC and about 160 mg THC per day, preferably between about 10 mg THC and about 100 mg THC per day per day; and
c) said method additionally comprising steps of administering said CBD or a derivative thereof in a dosage of about 2 mg CBD and about 40 mg CBD per day, preferably between about 2 mg CBD and about 20 mg CBD per day.
